(19) United States
(12) Patent Application Publication
(10) Pub. No.: US 2023/0404928 A1
(43) Pub. Date: Dec. 21, 2023

(54) NANOCAPSULE AND USES THEREOF

(71) Applicant: CHINA MEDICAL UNIVERSITY, Taichung (TW)

(72) Inventors: Woei-Cherng SHYU, HSINCHU CITY (TW); Chih-Sheng CHIANG, Taichung City (TW); Long-Bin JENG, Taichung City (TW); Yen-Ho LAI, HSINCHU CITY (TW); San-Yuan CHEN, HSINCHU (TW)

(21) Appl. No.: 18/335,897

(22) Filed: Jun. 15, 2023

Related U.S. Application Data
(60) Provisional application No. 63/366,465, filed on Jun. 15, 2022.

(51) Int. Cl. A61K 9/50; A61K 49/18; A61K 9/51; A61K 38/48; A61P 7/02

(52) U.S. Cl. CPC A61K 9/5036; A61K 49/1824; A61K 9/5123; A61K 9/5115; A61K 38/482; A61P 7/02; C12Y 304/21068

(57) ABSTRACT

The present disclosure provides a nanocapsule comprising a hydrophilic core; and a hydrophobic shell enclosing the hydrophilic core. The hydrophobic shell contains an outer layer comprising fucoidan, a middle layer comprising carotenoids and metal oxide nanoparticles, and an inner layer comprising fucoidan and contacting the hydrophilic core. The present disclosure also provides use of the nanocapsule as disclosed herein in the manufacture of a medicament for treating and/or diagnosing diseases in a subject in need thereof.

### CROSS-REFERENCE TO RELATED APPLICATION

This application claims the benefit to and priority of U.S. provisional application Ser. No. 63/366,465, filed Jun. 15, 2022, the full disclosure of which is incorporated herein by reference.

### FIELD OF THE INVENTION

The present disclosure relates to a therapeutic agent and method, more particularly to a therapeutic nanocapsule and uses thereof.

### BACKGROUND OF THE INVENTION

Stroke remains a leading cause of disability. Recombinant tissue plasminogen activator (tPA) is the only FDA-approved thrombolytic drug for patients with acute ischemic stroke. However, because of its short half-life, narrow therapeutic window, and high risk of intracranial hemorrhage, only a small population of stroke patients (1-2%) can benefit from its use. Moreover, inflammatory cells are postulated to mediate some of the brain damage. Additionally, oxidative stress is regarded as a major flexible operative agent in ischemic brain damage. These mechanisms can occur simultaneously or sequentially during the ischemic stroke. However, no single drug can target the multiple mechanisms at once; therefore, the therapeutic efficacy is limited.

There is thus a need for improved methods of treating thrombosis.

### SUMMARY OF THE INVENTION

The present disclosure provides a novel therapeutic agent for treating diseases.

In one embodiment of the present disclosure, the therapeutic agent is a nanocapsule comprising
a hydrophilic core; and
a hydrophobic shell enclosing the hydrophilic core, wherein the hydrophobic shell contains an outer layer comprising fucoidan, a middle layer comprising carotenoids and metal oxide nanoparticles, and an inner layer comprising fucoidan and contacting the hydrophilic core.

In one embodiment of the present disclosure, the hydrophilic core comprises tissue plasminogen activator (tPA).

Examples of the carotenoids include, but are not limited to lycopene, beta and alpha-carotene, lutein, astaxanthin (ASTX or AST), zeaxanthin, capsanthin, canthaxanthin, phytoene and phytofluene.

In one embodiment of the present disclosure, the metal oxide nanoparticles are iron oxide nanoparticles. Examples of the iron oxide include, but are not limited to Fe₃O₄ or Fe₂O₃.

In some embodiments of the disclosure, the weight ratio of fucoidan to carotenoids is from 0.55 to 110; from 0.6 to 100; from 0.7 to 95; from 0.8 to 90; from 0.9 to 85; from 1.0 to 80; from 1.1 to 75; from 1.2 to 70; from 1.3 to 75; from 1.35 to 60; from 1.37 to 55; from 1.4 to 50; from 1.5 to 40; from 2.0 to 30; from 2.1 to 25; from 2.2 to 22; from 2.5 to 20; from 3.0 to 15; from 3.5 to 10.

In some embodiments of the disclosure, the weight ratio of carotenoids to metal oxide nanoparticles is from 0.01 to 100; from 0.012 to 90; from 0.014 to 80; from 0.015 to 70; from 0.016 to 60; from 0.018 to 50; from 0.020 to 40; from 0.022 to 30; from 0.024 to 28; from 0.026 to 25; from 0.03 to 24; from 0.04 to 22; from 0.05 to 20; from 0.06 to 18; from 0.07 to 16; from 0.08 to 14; from 0.082 to 12; from 0.085 to 10; from 0.083 to 12; from 0.08 to 10; from 0.075 to 8; from 0.07 to 6; from 0.065 to 4; from 0.06 to 2.

In some embodiments of the disclosure, the nanocapsule is free of surface modifier, surfactant, agglomeration inhibitor, dispersion stabilizer and/or viscosity modifier.

In some embodiments of the disclosure, the carotenoids and metal oxide nanoparticles form non-covalent bonds.

In some embodiments of the disclosure, the carotenoids and fucoidan form hydrogen bonds.

In some embodiments of the disclosure, the nanocapsule further comprises iron oxide nanoparticles, gold nanoparticles, gadolinium-based materials, or isotopes.

The present disclosure also provides use of the nanocapsule as disclosed herein in the manufacture of a medicament for treating and/or diagnosing diseases in a subject in need thereof. Alternatively, the present disclosure provides a method for treating and/or diagnosing diseases in a subject in need thereof, comprising administering the subject with a therapeutically effective amount of the nanocapsule as disclosed herein to the subject.

Examples of the diseases include, but are not limited to cancers, thrombosis or hypercoagulation, inflammation, or rheumatoid arthritis. Examples of thrombosis or hypercoagulation include, but are not limited to atherosclerosis, ischemic stroke, and intracranial hemorrhage.

In some embodiments of the disclosure, the nanocapsule is guided by a magnetic device.

In some embodiments of the disclosure, a novel hollow core-shell nanostructure is manufactured by manipulating HLB value of blending compounds possessing different physiological properties. The hollow core can be used to encapsulate drug or therapeutic agents for different purposes. In some embodiments of the disclosure, the nanocapsule can be synthesized using fucoidan, ASTX, and iron oxide. Furthermore, the nanocapsule can be used to screen ideal compounds combined from the database to form a hollow core-shell structure possessing multiple functions. In addition, the combination of antioxidant and anti-inflammation building block materials along with ideal drugs can be extended to the application of multiple disease treatments in correlation with inflammation, cancer, rheumatoid arthritis, and atherothrombotic disease. In sum, the present disclosure provides: screening system for forming ideally inherently theranostic nanoparticles, fucoidan-ASTX nanostructures, and theranostic ability to treat stroke with significantly high probability to extend toward other applications.

These and other aspects will become apparent from the following description of the preferred embodiments taken in conjunction with the following drawings, although variations and modifications may be effected without departing from the spirit and scope of the novel concepts of the disclosure.

### BRIEF DESCRIPTION OF THE DRAWINGS

FIG. 1 shows a cross-sectional view of a nanocapsule in one embodiment of the disclosure.

FIGS. 2A to 2D show analysis of the nanocapsule (FANC). FIG. 2A: SEM image; FIG. 2B: TEM image; FIG. 2C: particle size analysis; and FIG. 2D: magnetophore curves.

FIGS. 3A to 3D show analysis of the control groups of 10-free fucoidan/astaxanthin nanoparticles (FANP) or astaxanthin-free fucoidan/IO nanoparticles (FFNP). FIG. 3A: SEM image of FANP; FIG. 3B: TEM image of FANP; FIG. 3C: SEM image of FFNP; FIG. 3D: TEM image of FFNP.

FIG. 4 shows drug release curves.

FIG. 5 shows FTIR of fucoidan, ASTX and FAFNC.

FIG. 6 shows that FANC mediated immunomodulation induces neuroprotection on ischemic brains. FANC (FA-NP) is shown a significant reduction in anti-inflammatory factors IL-10.

FIG. 7A shows a representative image of TUNEL staining of stroke brain. Cellular apoptosis was observed in MACO model mice after treatment with different groups in comparison to the control group. (Scale bar=50 μm). FIG. 7B shows the results of the representative image of TUNEL staining of stroke brain expressed as mean±SD., n=3 biologically independent samples. Statistical analysis was performed by Graph Pad Prism 9.0 Software; one-way ANOVA with Turkey correction, #P<0.05, ##P<0.01 between groups and two-sided t-test compared with saline-control, marked asterisks (*) with *P<0.05 and **P<0.01 representing statistically significant differences.

FIG. 8A shows a representative image of right MCA at pre-MCAO, post-MCAO and 4 h after each treatment stage in stroke mice. FIG. 8B shows Quantitative measurement of lysis time recorded from MACO mice in different treatment groups. FIG. 8C shows cortical blood flow (CBF) change plotted in accordance with the lysis time course after MACO in different treatment groups. FIG. 8D shows neurological behavior analysis including, number of vertical movements, vertical activity, and vertical movement time of MACO mice at 1, 7, 14, 28 days after injection with different groups in MACO model mice. The results are expressed as mean±SD., n=4 biologically independent animals. For FIGS. 8B, 8C and 8D, statistical analysis was performed by Graph Pad Prism 9.0 Software; two-way ANOVA with Turkey correction, #P<0.05, and #P<0.01, between groups and two-sided t-test compared with control, *p<0.05, and **p<0.01.

FIGS. 9A to 9D show representative images of Ki-67 staining after MTDS with MN treatment in comparison with saline control, free rt-PA, MTDS in SVZ area (FIG. 9A) and HDG area (FIG. 9B). (Scale bar=50 μm). Quantitative data of NPCs by immunohistochemistry staining in SVZ area (FIG. 9C) (Ki-67⁺ cells) and HDG area (FIG. 9D) (Ki-67⁺ cells).

### DETAILED DESCRIPTION OF THE INVENTION

### Definitions

Unless otherwise defined, all technical and scientific terms used herein have the same meaning as commonly understood by one of ordinary skill in the art to which this disclosure pertains. In the case of conflict, the present document, including definitions, will control. As used in this specification and the appended claims, the singular forms “a,” “an” and “the” include plural references unless the content clearly dictates otherwise.

Throughout this specification, unless the context requires otherwise, the word “comprise,” or variations such as “comprises” or “comprising,” will be understood to imply the inclusion of a stated element or integer or group of elements or integers but not the exclusion of any other element or integer or group of elements or integers.

As used herein, the term “nanocapsule” refers to a nano- or subnano-secondary structure. The nanocapsules have an average diameter of between about 100 nm and about 1000 nm, preferably between 100 and 900 nm, more preferably between about 100-300 nm and about 300-500 nm. Also, the size of the nanocapsules in the microspheres is essentially uniform with about 99% of the remains having a diameter of less than 1 micron. As used herein, the term “nanocapsule” should be understood to be synonymous with any structures containing a hydrophilic core and a hydrophobic shell enclosing the hydrophilic core.

The term “shell” in the context of the present disclosure denotes any solid or semi-solid polymeric structure enclosing the remains.

As used herein, “hydrophilic” refers to a substance or portion thereof that more readily associates with water than with organic solvent.

As used herein, “hydrophobic” refers to molecules which have a greater affinity for, or solubility in an organic solvent as compared to water.

As used herein, the terms “treatment” and “treating” embrace both preventative, i.e. prophylactic, or therapeutic, i.e. curative and/or palliative, treatment. Thus, the terms “treatment” and “treating” comprise therapeutic treatment of patients having already developed said condition, particularly in manifest form. Therapeutic treatment may be symptomatic treatment in order to relieve the symptoms of the specific indication or causal treatment in order to reverse or partially reverse the conditions of the indication or to stop or slow down the progression of the disease. Thus, the conjugates, compositions, and methods of the present disclosure may be used for instance as therapeutic treatment over a period of time as well as for chronic therapy. In addition, the terms “treatment” and “treating” comprise prophylactic treatment, i.e. a treatment of patients at risk to develop a condition mentioned hereinbefore, thus reducing said risk.

As used herein, the terms “diagnosis” refers to methods of estimating or determining whether or not a patient is suffering from a given disease or condition or severity of the condition. Diagnosis does not require ability to determine the presence or absence of a particular disease with 100% accuracy, or even that a given course or outcome is more likely to occur than not. Instead, the “diagnosis” refers to an increased probability that a certain disease or condition is present in the subject compared to the probability before the diagnostic test was performed. Similarly, a prognosis signals an increased probability that a given course or outcome will occur in a patient relative to the probability before the prognostic test.

As used herein, the term “therapeutically effective amount” means an amount of a conjugate of the present disclosure that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that includes the knowledge that the subject is ill, or will be ill, as the result of a condition that is treatable by the compounds of the present disclosure.

Nanocapsule

Particularly, the present disclosure provides a nanocapsule comprising
a hydrophilic core; and a hydrophobic shell enclosing the hydrophilic core, wherein the hydrophobic shell contains an outer layer comprising fucoidan, a middle layer comprising carotenoids and metal oxide nanoparticles, and an inner layer comprising fucoidan and contacting the hydrophilic core.

In some embodiments, the nanocapsule may be formed from emulsions having hydrophilic-phase droplets dispersed in a hydrophobic substance. One example is water-in-oil emulsions. Water-in-oil emulsions include hydrophilic-phase droplets (e.g., as the dispersed phase) dispersed in the hydrophobic phase (e.g., as the continuous phase). If a compound (active substance) is hydrophilic, or it can be dissolved or dispersed in a hydrophilic solvent (e.g. water), then it is possible to encapsulate it in hydrophilic- (e.g., water-) core microcapsules. When a compound does not have sufficient solubility in the hydrophilic solvent, a co-solvent may be used to improve the dissolution of the compound and to facilitate the encapsulation process. Similarly, when a compound cannot be dispersed into the hydrophilic phase to form a reasonably stable suspension (e.g., indicated by droplets of the compound being dispersed throughout the hydrophilic phase and the compound remaining dispersed during emulsion formation and encapsulation processes).

In some embodiments, the nanocapsule is typically used for encapsulating water-soluble materials, but not oil-soluble materials, such as non-polar molecules. Oil-soluble materials can be incorporated into hydrophilic-core microcapsules by first adding them to a co-solvent, and then adding the resulting solution to the hydrophilic phase. Alternatively, a surfactant can be added to the hydrophilic phase. This will dissolve or disperse the non-polar or oil-soluble reagents into the hydrophilic phase. The emulsion (e.g. water-in-oil emulsion) can then be formed by adding the hydrophilic phase to a hydrophobic phase and a reaction can be initiated to encapsulate the oil, with the active substance dissolved or dispersed therein, into the core of the hydrophilic-core microcapsules.

Examples of the hydrophilic core include, but are not limited to, an anticancer drug, an anti-inflammation drug, a drug for stroke medication, an immune modulator, a nucleic acid molecule, an antibacterial drug, an antiviral drug, an anticoagulant drug, or an antioxidant drug.

Examples of the anticancer drug include, but are not limited to, bleomycin, cisplatin, carboplatin, cytarabine, docetaxel, doxorubicin, daunorubicin, epirubicin, fluorouracil, gemcitabine, irinotecan, leuprorelin, oxaliplatin, paclitaxel, pemetrexed, topotecan, vinorelbine, or vinblastine.

Examples of the anti-inflammation drug include, but are not limited to, ibuprofen, naproxen sodium, diclofenac potassium, celecoxib, sulindac, oxaprozin, piroxicam, or indomethacin.

Examples of the drug for stroke medication include, but are not limited to, tissue plasminogen activator, warfarin, clopidogrel, aspirin, atorvastatin, fluvastatin, lovastatin, pitavastatin, pravastatin, rosuvastatin, or simvastatin.

Examples of the immune modulator include, but are not limited to, cytokines, thalidomide, lenalidomide, pomalidomide, or imiquimod.

Examples of the nucleic acid include, but are not limited to, messenger RNA (mRNA), RNA inhibitor (RNAi), or microRNA.

In some embodiments, the hydrophobic shell substantially encloses or encapsulates the hydrophilic core. The hydrophobic shell contains an outer layer forming the outermost cover or surface of the nanocapsule. Particularly, the hydrophobic shell comprises fucoidan. Fucoidan, a sulfated polysaccharide found mainly in various species of edible seaweed offers several potentially beneficial bioactive functions for humans. Fucoidan possesses multiple biological activities including antibacterial, antiviral, antitumor, anticoagulant, and antioxidant activities. Fucoidan is a specific competitive inhibitor of p-selectin and L-selectin that can target and destroy platelets within the thrombus or labeled with isotopes as an imaging biomarker for assessing p-selectin activation in ischemic stroke. Blocking the selectin-mediated tethering step may limit the inflammatory component of reperfusion injury in the brain. Fucoidan is more specific anti-inflammatory agents and has been reported to decrease leukocyte accumulation during reperfusion of stroke. Selectin blockade significantly reduced cerebral infarction size and improved neurological function. In addition, a trend toward decreased cerebral edema was demonstrated with selectin inhibition.

In some embodiments of the disclosure, fucoidan is produced by Fucus vesiculosus, Okinawa mozuku, Cladosiphon okamuranus Tokida, and Undaria pinnatifida, and can be purified or partially purified from culture of the organisms. In some embodiments of the disclosure, fucoidan has a peak molecular ranged from 500 Da to 500 kDa; from 750 Da to 450 kDa; from 1 kDa to 400 kDa; from 50 kDa to 350 kDa; from 100 kDa to 300 kDa; from 150 kDa to 250 kDa; from 200 kDa to 225 kDa.

In some embodiments, the hydrophobic shell contains a middle layer beneath the outer layer. The middle layer comprises carotenoids and metal oxide nanoparticles.

As used herein, “xanthophylls” refer to hydroxy- and keto-oxidized carotenes and their derivatives, including both free alcohols and esters; “carotenes” refer to any of the 40-carbon carotenes and their derivatives; “retinoids” refers to the 20-carbonVitamin A (retinol) and its derivatives; and “carotenoids” refers to any of the xanthophylls, carotenes and retinoids or combinations thereof. Carotenoids may be synthetically derived or purified from natural sources. Synthetic preparations may contain different isomers of carotenoids than those contained in the natural preparations. Examples of the carotenoids include, but are not limited to lycopene, beta and alpha-carotene, lutein, astaxanthin, zeaxanthin, capsanthin, canthaxanthin, phytoene and phytofluene. In some embodiments, the carotenoids are astaxanthin. Astaxanthin, a lipid-soluble pigment can reduce reactive oxidizing molecules to be responsible for the antioxidant function. ASTX increased the activity of catalase, superoxide dismutase, and glutathione peroxidase as well as decreased the content of malondialdehyde in brain tissue. Importantly, astaxanthin turns on the expression of brain-derived neurotrophic factor (BDNF) and nerve growth factor (NGF) which further improve the stem cells potency through an increase in the proliferation of neural progenitor cells (NPCs).

Not limited by theory, it is believed that ASTX further increases the structure stability of nanocapsule with the specific range of quantity. By manipulating the ratio of molar concentrations between ASTX and species-specific fucoidan with appropriate molecular weight, a hydrophilic lipophilic balance value can be obtained to form nanoscale FANP with hollow core-shell structure.

In another aspect, ASTX has been found to possess substantial anti-oxidation activity. ASTX reduced the inflammation and myocardial injury of ischemia/reperfusion, alleviating the tissue damage associated with reperfusion injury. However, ASTX is a highly unsaturated molecule that tends to chemical degradation (oxidation) when exposed to high temperatures, oxygen, light, and pH extremes. The oxidative degradation of ASTX resulted in a loss of biological activity. Not limited by theory, it is believed that the nanocapsule shows protective function to prevent ASTX from oxidation through physiochemical manners. Facilitating the unique structure and formulation design, ASTX showed significantly improved self-antioxidant properties, which subsequently provide a durable therapeutic effect at its desire site of action. In some embodiments of the disclosure, the nanocapsule can retain the 50% non-oxidation level of ASTX for 15 days in solution and over 3 months in powder form, which is significantly longer than that of free ASTX form.

In certain embodiments, the metal oxide nanoparticles comprise iron oxide including, but not limited to Fe₃O₄ or Fe₂O₃. Alternatively, the metal oxide can also comprise cobalt, magnesium, samarium, zinc, or mixtures of these and other metals with or without iron. The metal oxide core can be magnetic, paramagnetic and superparamagnetic. The term “magnetic” means materials of high positive magnetic susceptibility. In one embodiment, a superparamagnetic form of iron oxide is used. Superparamagnetic iron oxide is one of the highly magnetic forms (magnetite, non-stoichiometric magnetite, gamma-ferric oxide) that have a magnetic moment of greater than about 30 EMU/gm Fe at 0.5 Tesla and about 300 K. When the magnetic moment is measured over a range of field strengths, it shows magnetic saturation at high fields and lacks magnetic remanence when the field is removed.

In some embodiments of the disclosure, the weight ratio of carotenoids to metal oxide nanoparticles is from 0.55 to 110; from 0.6 to 100; from 0.7 to 95; from 0.8 to 90; from 0.9 to 85; from 1.0 to 80; from 1.1 to 75; from 1.2 to 70; from 1.3 to 75; from 1.35 to 60; from 1.37 to 55; from 1.4 to 50; from 1.5 to 40; from 2.0 to 30; from 2.1 to 25; from 2.2 to 22; from 2.5 to 20; from 3.0 to 15; from 3.5 to 10.

In some embodiments, the hydrophobic shell contains an inner layer beneath the middle layer and contacting the hydrophilic core. Particularly, the hydrophobic shell comprises fucoidan.

In some embodiments of the disclosure, the weight ratio of fucoidan to carotenoids is from 0.01 to 100; from 0.012 to 90; from 0.014 to 80; from 0.015 to 70; from 0.016 to 60; from 0.018 to 50; from 0.020 to 40; from 0.022 to 30; from 0.024 to 28; from 0.026 to 25; from 0.03 to 24; from 0.04 to 22; from 0.05 to 20; from 0.06 to 18; from 0.07 to 16; from 0.08 to 14; from 0.082 to 12; from 0.085 to 10; from 0.083 to 12; from 0.08 to 10; from 0.075 to 8; from 0.07 to 6; from 0.065 to 4; from 0.06 to 2.

In some embodiments of the disclosure, the nanocapsule is free of a surface modifier, surfactant, agglomeration inhibitor, dispersion stabilizer and/or viscosity modifier. Conventionally, a surfactant must be used to improve the dispersion of the compound and facilitate the encapsulation process, such that if a compound can be dissolved or dispersed in a hydrophilic solvent, with or without the aid of a co-solvent or a surfactant, it is possible to encapsulate it into hydrophilic-core microcapsules. On the contrary, the hydrophilic core and hydrophobic shell are formed without surface modifier, surfactant, agglomeration inhibitor, dispersion stability.

Not limited by theory, it is believed that the metal oxide nanoparticles located in the two interfaces are able to adsorb the water-oil interface. Through the metal oxide nanoparticles, the interfacial energy can be reduced, so the required interfacial active agent can be reduced in the process of forming the nanocapsule. The nanocapsule has the following advantages: the amount of emulsifier can be greatly reduced; the toxicity to human body is much less than that of surface modifier; the emulsion is stable and not easily affected by the pH, salt concentration, temperature and oil phase composition of the system.

The surfactant may be, for example, non-ionic surfactant including polyoxyethylene (hereinafter referred to as “POE”)-polyoxypropylene (hereinafter referred to as “POP”) block copolymers such as poloxamer 407, poloxamer 235 and poloxamer 188; ethylenediamine adducts to polyoxyethylene-polyoxypropylene block copolymer such as poloxamine; POE sorbitan fatty acid esters such as POE (20) sorbitan monolaurate (polysorbate 20), POE (20) sorbitan monooleate (polysorbate 80) and polysorbate 60; POE hydrogenated castor oils such as POE (60) hydrogenated castor oil; POE alkyl ethers such as POE (9) lauryl ether; POE-POP alkyl ethers such as POE (20) POP (4) cetyl ether; POE alkylphenyl ethers such as POE (10) nonyl phenyl ether; POE-POP glycols such as POE (105) POP (5) glycol, POE (120) POP (40) glycol, POE (160) POP (30) glycol, POE (20) POP (20) glycol, POE (200) POP (70) glycol, POE (3) POP (17) glycol, POE (42) POP (67) glycol, POE (54) POP (39) glycol and POE (196) POP (67) glycol; amphoteric surfactants including glycine-type surfactants such as alkyldiaminoethyl glycine, betaine acetate-type surfactants such as lauryl dimethylaminoacetic acid betaine, and imidazoline-type surfactants; anionic surfactants including POE alkyl ether phosphates and salts thereof such as POE (10) sodium lauryl ether phosphate, N-acylamino acid salts such as sodium lauroyl methyl alanine, alkyl ether carboxylates, N-acyl taurates such as sodium cocoyl N-methyltaurate, sulfonates such as sodium tetradecenesulfonate, alkyl sulfates such as sodium lauryl sulfate, POE alkyl ether sulfates such as POE (3) sodium lauryl ether sulfate, and α-olefin sulfonates; and cationic surfactants including alkylamine salts, alkyl quarternary ammonium salts (benzalkonium chloride and benzethonium chloride) and alkyl pyridinium salts (cetylpyridinium chloride and cetylpyridinium bromide).

The agglomeration inhibitor may be phospholipids such as alkyl sulfate, N-alkyloyl methyl taurate, ethanol, glycerol, propylene glycol, sodium citrate, phospholipids including glycerophospholipid (lecithin (phosphatidylcholine) (e.g., refined soybean lecithin, hydrogenated soybean lecithin), phosphatidylserine, phosphatidylethanolamine, phosphatidylinositol, phosphatidic acid, phosphatidylglycerol, lysophosphatidylcholine, lysophosphatidylserine, lysophosphatidylethanolamine, lysophosphatidylinositol, lysophosphatidic acid and lysophosphatidylglycerol) and sphingophospholipids (sphingomyelin, ceramide, glycosphingolipid or ganglioside), D-sorbitol, lactose, xylitol, gum arabic, sucrose fatty acid ester, polyoxyethylene hydrogenated castor oil, polyoxyethylene fatty acid esters, polyethyleneglycol (PEG), polyoxyethylene sorbitan fatty acid ester, alkyl benzene sulfonate, sulfosuccinate, POE-POP glycol, polyvinylpyrrolidone, PVA, hydroxypropyl cellulose, methyl cellulose, hydroxyethyl cellulose, hydroxypropyl methylcellulose, carmellose sodium, carboxyvinyl polymers, N-acyl-glutamate, acrylic acid copolymers, methacrylic acid copolymers, casein sodium, L-valine, L-leucine, L-isoleucine, benzalkonium chloride and benzethonium chloride.

The “viscosity modifier” is capable of adjusting the viscosity of the aqueous suspension. The viscosity modifier may be polysaccharides or derivatives thereof (gum arabic, gum karaya, xanthan gum, carob gum, guar gum, gum guaiac, quince seed, darman gum, gum tragacanth, benzoin rubber, locust bean gum, casein, agar, alginic acid, dextrin, dextran, carrageenan, gelatin, collagen, pectin, starch, polygalacturonic acid, chitin and derivatives thereof, chitosan and derivatives thereof, elastin, heparin, heparinoid, heparin sulfate, heparan sulfate, hyaluronic acid and chondroitin sulfate), ceramide, cellulose derivatives (methyl cellulose, ethyl cellulose, hydroxyethyl cellulose, hydroxypropyl cellulose, hydroxypropyl methylcellulose, carboxymethyl cellulose, carboxyethyl cellulose, cellulose and nitrocellulose), PVA (completely or partially saponified), polyvinylpyrrolidone, Macrogol, polyvinyl methacrylate, polyacrylic acid, carboxyvinyl polymer, polyethyleneimine, polyethylene oxide, polyethylene glycol, ribonucleic acid, deoxyribonucleic acid, methyl vinyl ether-maleic anhydride copolymers, and pharmacologically acceptable salts thereof (e.g., sodium alginate). The aqueous suspension may contain one or two or more viscosity modifiers. The viscosity modifier is preferably one or more substances selected from hydroxypropyl methylcellulose (e.g., TC-5(R), Metlose 60SH-50), PVA (Kurary POVAL 217C) and methyl cellulose (e.g., Metlose SM-100, Metlose SM-15).

The “surface modifier” herein refers to the surfactant, the agglomeration inhibitor, the viscosity modifier and/or the dispersion stabilizer, and the surface modifier is capable of modifying the nanoparticle surface of clobetasol propionate.

The “dispersion stabilizer” usable herein is listed above as the surfactants, agglomeration inhibitors and/or viscosity modifiers, and is preferably one or more substances selected from polyoxyethylene hydrogenated castor oil 60, polyoxyethylene hydrogenated castor oil 40, polysorbate 80, polysorbate 20, POE-POP glycol, PVA, hydroxypropyl methylcellulose and methyl cellulose, and more preferably one or more substances selected from POE-POP glycol, PVA, hydroxypropyl methylcellulose and methyl cellulose.

In some embodiments of the disclosure, the carotenoids and metal oxide nanoparticles form non-covalent bonds.

In some embodiments of the disclosure, the carotenoids and fucoidan form hydrogen bonds.

In some embodiments of the disclosure, the nanocapsule further comprises iron oxide nanoparticles, gold nanoparticles, gadolinium-based materials or isotopes.

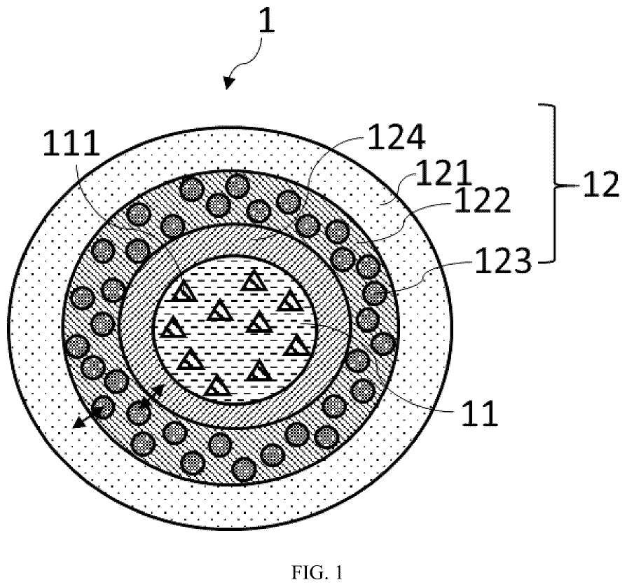

Referring to FIG. 1 representing a nanocapsule 1 in one embodiment of the disclosure. The nanocapsule 1 comprises a hydrophilic core 11 containing tPA 111. The nanocapsule 1 comprises a hydrophobic shell 12 enclosing the hydrophilic core 11. The hydrophobic shell 12 contains an outer layer 121 comprising fucoidan, a middle layer 122 comprising carotenoids 123 and metal oxide nanoparticles, and an inner layer 124 comprising fucoidan and contacting the hydrophilic core 11. Arrows indicate hydrogen bonds formed between the carotenoids and fucoidan.

Therapeutic Uses

The present disclosure also provides use of the nanocapsule as disclosed herein in the manufacture of a medicament for treating and/or diagnosing diseases in a subject in need thereof. Alternatively, the present disclosure provides a method for treating and/or diagnosing diseases in a subject in need thereof, comprising administering the subject with a therapeutically effective amount of the nanocapsule as disclosed herein to the subject.

Examples of the diseases include, but are not limited to cancers, thrombosis or hypercoagulation, inflammation, or rheumatoid arthritis.

As used herein, thrombosis refers to a thrombus (blood clot) inside a blood vessel. The term encompasses, without limitation, arterial and venous thrombosis, including deep vein thrombosis, portal vein thrombosis, jugular vein thrombosis, renal vein thrombosis, stroke, myocardial infarction, Budd-Chiari syndrome, Paget-Schroetter disease, and cerebral venous sinus thrombosis. Diseases and conditions associated with thrombosis and the risk of developing thrombosis or hypercoagulation include, without limitation, acute venous thrombosis, pulmonary embolism, thrombosis during pregnancy, hemorrhagic skin necrosis, acute or chronic disseminated intravascular coagulation (DIC), clot formation from surgery, long bed rest, long periods of immobilization, conditions that preclude or restrict movement such as partial or complete paralysis, morbid obesity, disorders that impede oxygen uptake and absorption such as lung disorders including lung cancer, COPD, emphysema, drug related fibrosis, cystic fibrosis, venous thrombosis, fulminant meningococcemia, acute thrombotic stroke, acute coronary occlusion, acute peripheral arterial occlusion, massive pulmonary embolism, axillary vein thrombosis, massive iliofemoral vein thrombosis, occluded arterial cannulae, occluded venous cannulae, cardiomyopathy, venoocclusive disease of the liver, hypotension, decreased cardiac output, decreased vascular resistance, pulmonary hypertension, diminished lung compliance, leukopenia, and thrombocytopenia. Particularly, the thrombosis or hypercoagulation is atherosclerosis, ischemic stroke, or intracranial hemorrhage.

Intravenous administration of recombinant tissue plasminogen activator (rtPA) is the mainstream FDA approved thrombolytic treatment for emergent ischemic stroke. However, only a small population of patients can benefit from the medicine. Furthermore, fucoidan and ASTX represent potential compounds to treat ischemic stroke. However, properties such as solubility in aqueous solution, oxidative nature, and molecular weight have hindered the therapeutic benefit due to unavailability of reaching the disease niche. In some embodiments of the disclosure, the nanocapsule comprises rtPA in the hydrophilic core. The core-shell structure is only available when a specific range of ratio between ASTX and fucoidan of particular species and molecular weight. In some embodiments, the nanocapsule (FANC) shows the ability to target thrombolytic and protect neurons in the ischemic stroke model due to the function of fucoidan and ASTX. In addition, by carrying rtPA, the nanocapsule (rtPA@FANC) shows capacity to achieve marked enhancement on preventing rapid metabolism was observed, which in turns significantly extended median survival in vivo. In sum, the nanocapsule as a theranostic nanostructure can be obtained within a specific synthesis condition for targeting p-selectin/CXCR4 and treating atherothrombotic disease.

In some embodiments of the disclosure, rtPA is encapsulated to treat patient with stroke. The rtPA-loaded FANC (rtPA@FANC) possesses multiple functions including platelets targeting, p-selectin blockade, anti-inflammation, ROS attenuation, and induction of stem cells potency as well as the therapeutic efficacy resulted from rtPA within the single nanosystem. In other words, rtPA@FANC can target multiple pathways of stroke for augmenting therapeutic outcome. Moreover, induction of endogenous NPCs proliferation after rtPA@FANC might play an important role to repair the injury neurons in this ischemic environment. In other words, in current treatment, there is no such drug or mechanism that can treat stroke in multiple aspects. Thus, rtPA@FANC represents the first-in-class strategy to treat all pathology pathways of stroke with all-in-one nanostructure system.

In some embodiments, the nanocapsule can be used as a contrast agent, drug, and the combination theranostic agent for monitoring/treating diseases (e.g., cancers, thrombosis or hypercoagulation, inflammation, or rheumatoid arthritis) that presents p-selectin/CXCR4.

Not limited by theory, it is believed that the iron oxide nanoparticle (IO), gold nanoparticle, gadolinium-based materials, other metal/metal oxide nanoparticle or isotopes can further be incorporated in the nanocapsule to provide the imaging capacity (i.e., computer tomography, magnetic resonance imaging, single photon emission computed tomography, and positron emission tomography).

In some embodiments of the disclosure, the nanocapsule is guided by a magnetic device.

The following examples are provided to aid those skilled in the art in practicing the present disclosure.

### Examples

Nanocapsule

Abbreviation:
IO-free fucoidan/astaxanthin nanocapsule: FANP
Astaxanthin-free fucoidan/IO nanocapsule: FFNP
Fucoidan/astaxanthin with IO nanocapsule: FAFNC or FANC
recombinant tissue-type plasminogen activator: rtPA
Fucoidan/astaxanthin with IO nanocapsule carrier with rtPA: MTDS
M=magnet targeting

The synthesis of FANC and FANP or FFNP involves one organic phase (chloroform or dichloromethane) and two hydrophilic phases (double distilled water, DDW). A first hydrophilic phase containing fucoidan with rtPA (for MTDS, with a weight ratio of 0.085 to 10) or without rtPA (for FANC, FANP, and FFNP) in water (50 μl to 200 μl) was added with an organic phase containing IO and AST in chloroform or dichloromethane (300 μl to 500 μl). Then, these two phases were emulsified by pulsed ultrasound sonification (35 w, 30 seconds to 120 seconds) or nanoprecipitation (mixing and rotating for 0.5 h to 3 h) to form a W/O solution. Next, a second hydrophilic phase containing 2 ml of fucoidan was emulsified (70 w, 30 seconds to 180 seconds) or precipitated (mixing and rotating for 0.5 h to 3 h) with the W/O solution to obtain a final W/O/W nanocapsules or nanoparticles. The organic solvent was removed by evaporation (27° C., 30 min, 50 mmHg to 250 mmHg) or tangential flow filtration (TFF). The nanoparticles or nanocapsules were further purified with a magnetic selection equipment. At last, the nanoparticles or nanocapsule were re-suspended in a water solution (Ex distilled deionized water, containing saline or 5% glucose).

A nanocapsule of fucoidan/astaxanthin/IO nanocapsule (FANC) can be prepared using a double emulsification method. Firstly, the hydrophobic interaction of astaxanthin with iron oxide forms a non-covalently bonded core-shell structure, and astaxanthin provides a hydroxyl group that forms a hydrogen bond with a sulfate group in fucoidan, thus stabilizing the nanoparticles (as shown in FIG. 1). The structure protects the rtPA in FANC.

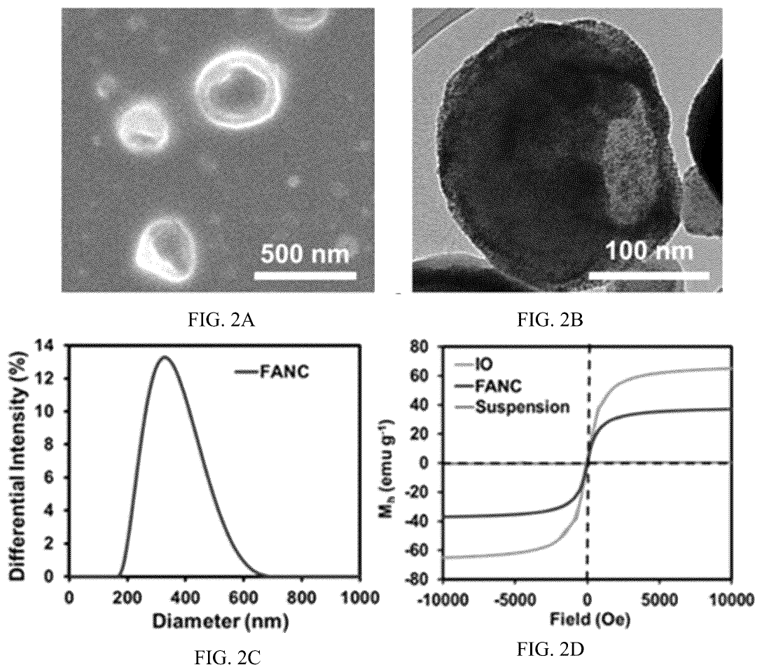

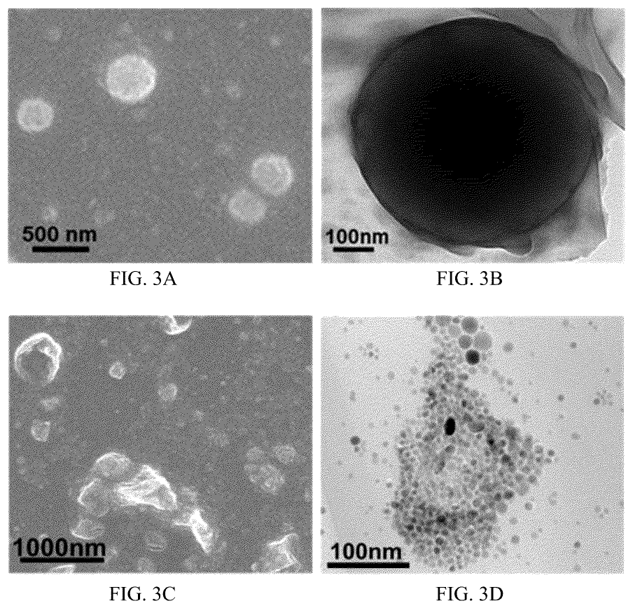

The morphology of FANC was analyzed with scanning electron microscopy (SEM) and transmission electron microscopy (TEM). The SEM image in FIG. 2a shows the folded structure of FANC, which can be attributed to the collapse of the core under vacuum conditions during imaging. The TEM image in FIG. 2b shows the core-shell structure of FANC with IO nanoparticles overlapping on the shell layer of FANC, creating a light-dark contrast. Control groups of IO-free fucoidan/astaxanthin nanoparticles (FANP) or astaxanthin-free fucoidan/IO nanoparticles (FFNP) are also provided. As shown in FIGS. 3a and 3b, FANP exhibited a solid structure with no space for loading rtPA. In the absence of surfactants and iron oxide, astaxanthin fails to form an oil-covered water-stable inner layer (w/o emulsion). On the other hand, although supported by the rigid structure of the iron oxide, the astaxanthin-free fucoidan/IO nanoparticles cannot form a stable nanocapsule because of lacking the hydrogen bonding force of the astaxanthin (FIGS. 3c and 3d). Based on the images under the SEM and TEM electron microscopy, broken carriers and randomly dispersed iron oxides are clearly observed, proving the lack of instability and infeasibility.

In addition, the particle size and mean distribution of FANC was measured by particle size analyzer, showing that the hydrated diameter is about 331 nm with homogeneous distribution (PDI <0.3) (FIG. 2c). The surface charge (zeta potential) of FANC was measured as −51.34±5.8 mV. This high negative charge can be attributed to the negatively charged sulfate in the fucoidan molecules, which is favorable for the dispersion and biocompatibility of the nanocapsule in the blood. To further confirm the function of magnetic particles in IO, the magnetization strengths of FANC and IO were measured at 64.9 and 37.1 emug-1, respectively, indicating that FANC still has magnetic properties (FIG. 2d). Thus, FANC can be purified and directed to the thrombus region by magnetic use of MagniSort® and IO.

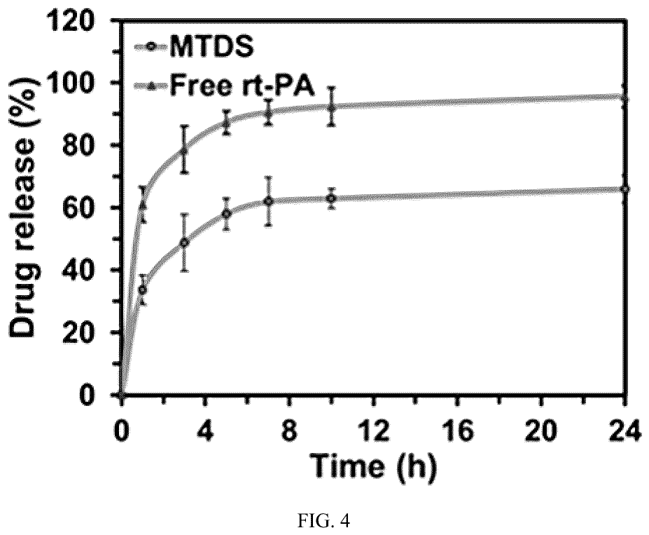

To measure the loading of rtPA (rtPA-FANC=(MTDS)) in FANC, analysis using Bradford protein analysis (BCA) was performed. The encapsulation efficiency (EE) of rtPA in FANC is 73.94% and the loading capacity (LC) is 22.49%. To observe the rtPA release rate of FANC, further drug release behavior studies were conducted. As shown in FIG. 4, the free rtPA shows a fast release rate, with more than 60% release in the first hour. In contrast, MTDS shows a slower release behavior due to the IO and ASTX shell structure as a diffusion barrier for rtPA, with only 58% of rtPA released cumulatively at 5 hours. This slower release behavior can effectively protect rtPA from metabolic effects and reduce the damage caused in the blood circulation system.

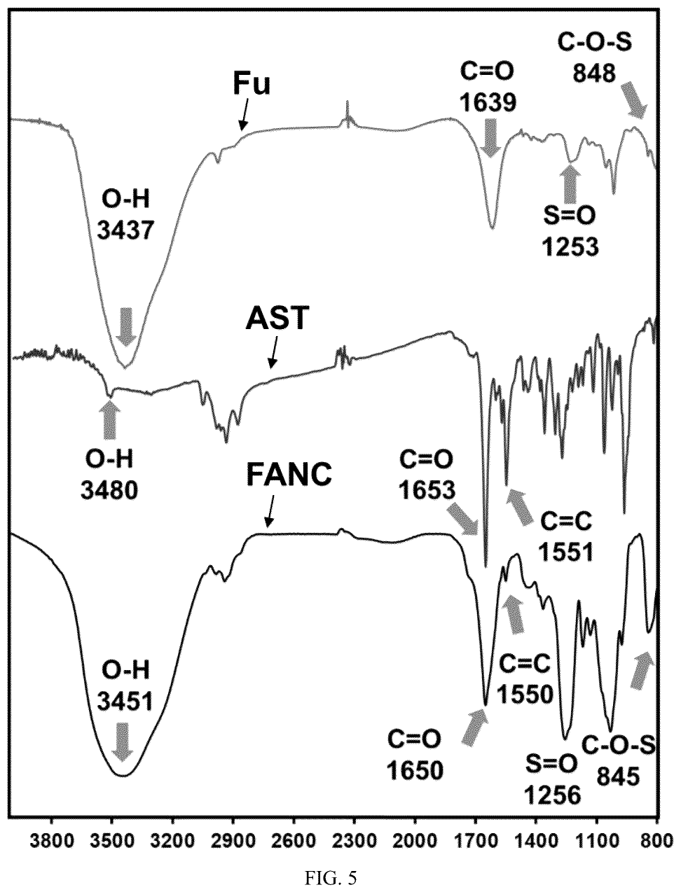

In addition, to verify the composition of the nanocapsule, the chemical structure of FANC was investigated using Fourier transform infrared spectroscopy (FTIR). As shown in FIG. 5, FANC has functional groups of fucoidan and ASTX, such as sulfate group in fucoidan (S═O, 1253 and 1256 cm⁻¹) and olefin group in ASTX (C═C, 1550 and 1551 cm⁻¹). In addition, the peaks at 845 and 848 cm⁻¹ indicate C—O—S bonds and are also designated as the signature functional groups of fucoidan. These results confirm the chemical composition of FANC as fucoidan and ASTX.

Therapeutic Uses

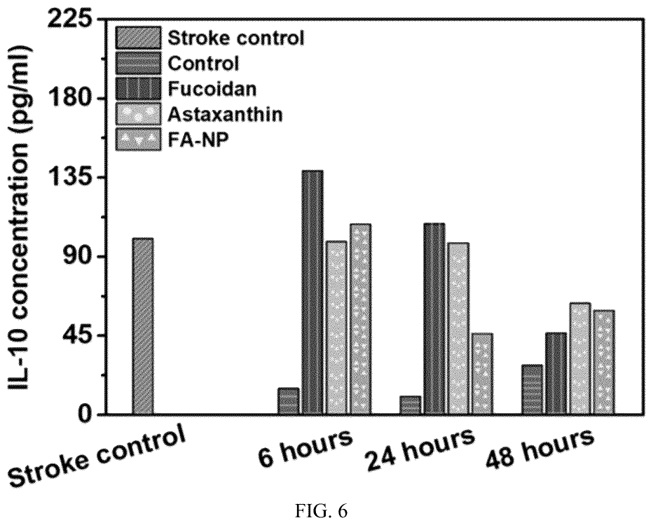

MTDS has multiple functions, including platelet targeting, p-selectin blockade, anti-inflammation, ROS mitigation and induction of stem cell potential, as well as therapeutic effects from rtPA, all in a single nanosystem. In other words, rtPA-FANC can target multiple pathways of stroke and enhance therapeutic efficacy. In addition, the induction of endogenous neural precursor cell proliferation of rtPA@FANC may play an important role in the repair of damaged neurons in the ischemic environment (FIG. 6). The nanocapsule or the mechanism thereof is novel for the treatment of stroke in multiple ways.

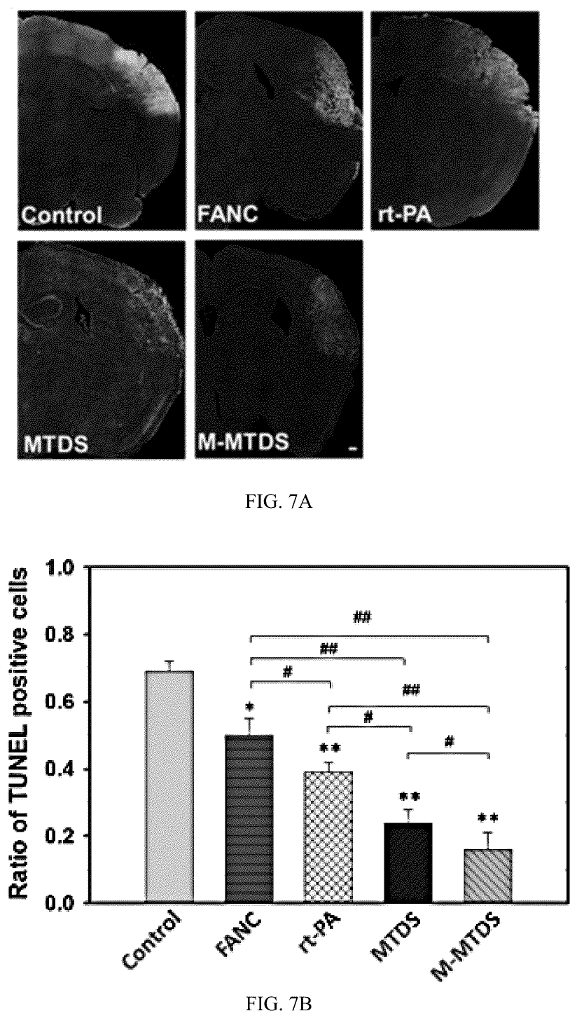

Apoptosis in the brain of stroke rats was assayed by TUNEL staining. In the absence of rtPA, FANC still exhibits some therapeutic effect (fewer TUNEL+ cells) compared to the control group (FIGS. 7a and 7b). This can be attributed to the therapeutic activity of FANC itself, as fucoidan has anti-inflammatory and neuroprotective effects by inhibiting NF-κB, MAPK and Akt activation. In addition, according to its chemical structure, ASTX is able to maintain the structural integrity of the cell membrane. Thus, the combined treatment of these two substances is effective in reducing cell death in stroke mice. MTDS-treated mice containing MN had fewer TUNEL+ cells in the semidark zone around the ischemic nucleus than the other four groups of animals. These results suggest that the dual-targeted delivery system can not only carry rtPA for emergency treatment of stroke mice, but also provide anti-inflammatory and neuroprotective effects through FANC itself.

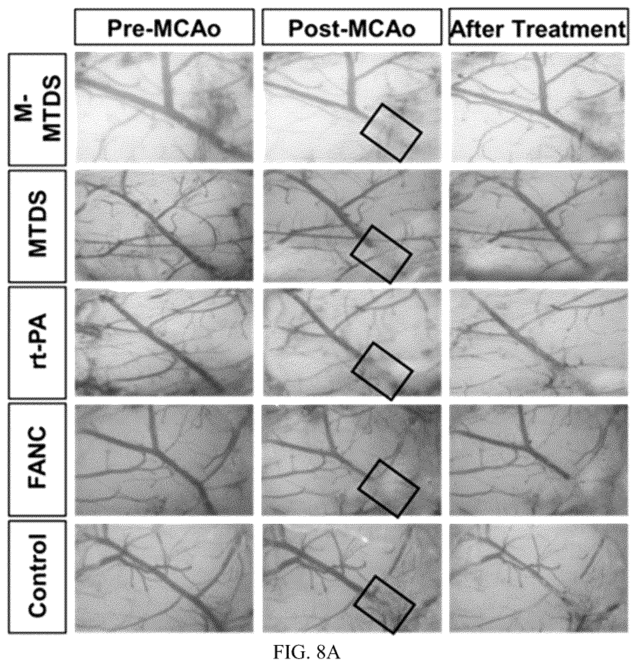

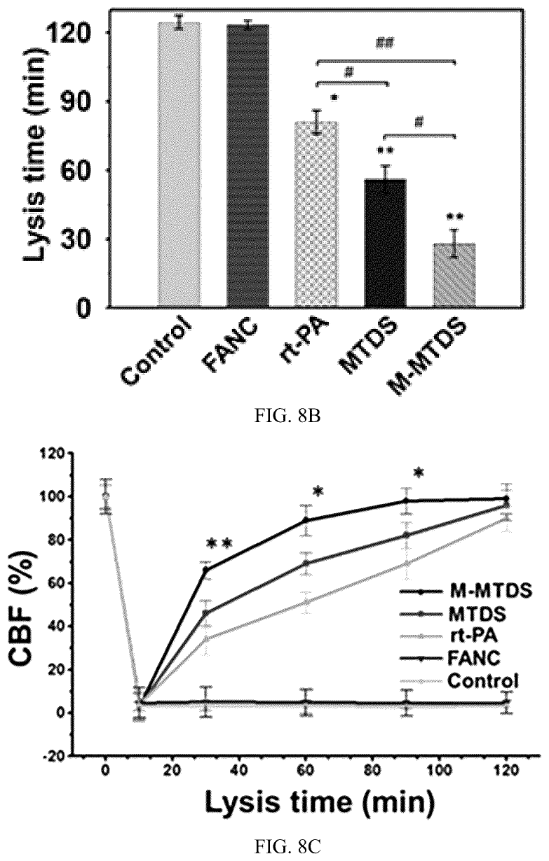

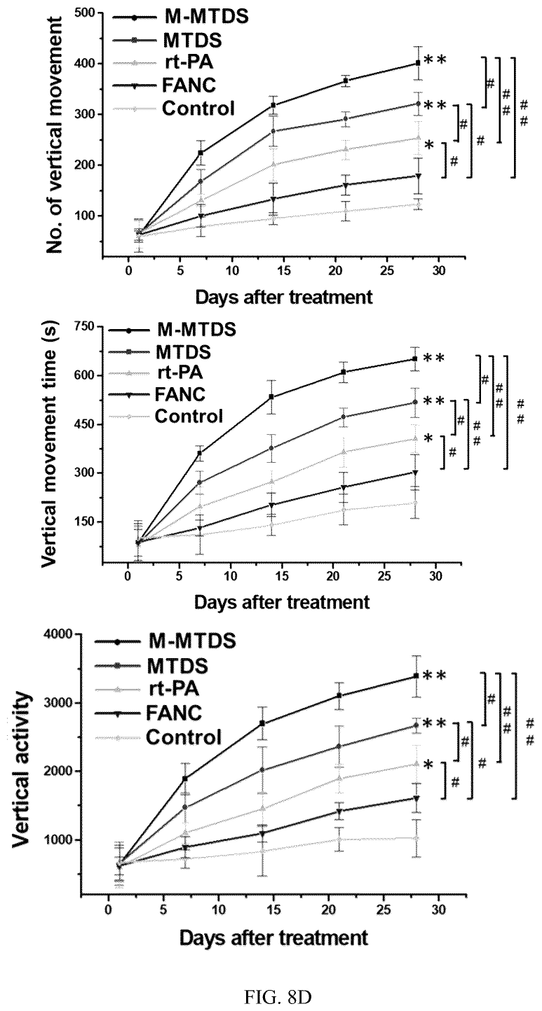

The efficacy of the nanocapsule in a rat model of ischemic stroke was assayed. FIG. 8a shows the thrombolytic activity of the different treatment groups, indicating that rt-PA, MTDS and MTDS with magnetic navigation (MN) (M-MTDS) groups are able to dissolve the thrombus. To investigate the effects of FANC and M-MTDS, cerebral blood flow and thrombolysis time were measured in each treatment group (FIGS. 8b and 8c). The results show that no thrombolytic effect was observed in the saline control and FANC groups at 120 minutes after MCAO. In contrast, rtPA (10 mg kg⁻¹) induced partial thrombolysis at approximately 80 minutes and restored blood flow (approximately 80%) within 120 minutes (FIGS. 8b-8c). However, rtPA was unable to completely dissolve the thrombus located in the aorta (CBF did not recover more than 90%). Importantly, the MTDS group was able to dissolve the thrombus within 60 minutes and recovered more than 80% of the CBF within 90 minutes, and such effects may result from the p-selectin targeting ability of fucoidan. In addition, the MTDS and M-MTDS groups show the shortest time to thrombolysis (within 30 minutes) and the fastest time to CBF recovery (more than 90% within 60 minutes). This improvement can be attributed to the FANC with magnetic navigation, which can significantly improve the thrombolytic efficiency and shorten the time of delivery to the brain. The results show that only a smaller dose of rtPA (one-fifth) is sufficient to achieve a higher thrombolytic efficiency. In addition, the recanalization time is significantly shorter compared to the rtPA treatment group. With stronger thrombolytic capacity and faster CBF recovery, MTDS and M-MTDS can be more effective in reducing ischemic damage caused by stroke.

To assess the neuroprotective effect on ischemic brain, the functional recovery of MCAO mice after each treatment was further analyzed and neurobehavioral measurements were recorded at 1, 7, 14, and 28 days after treatment (FIG. 8d). Motor activity (vertical activity, vertical time and number of vertical movements) was significantly better in mice treated with MTDS and M-MTDS after ischemic brain injury (FIG. 8d). In particular, the FANC group outperformed the control group without any of the therapeutic drugs carried. The results not only show significant improvement in neurological deficits caused by ischemic cerebral ischemia in the MTDS and M-MTDS groups, but also demonstrated the neuroprotective capacity of FANC. These results can be attributed to the combined recruitment and proliferation of neural precursor cells by fucoidan and ASTX.

The subventricular zone (SVZ) and hippocampal dentate gyrus (DG) are known to trigger the activation of resting neural precursor cells (NPCs) when the brain suffers from ischemic injury. NPCs mainly reside in the SVZ, while the DG has the ability to differentiate into a range of neurons and glial cells. To demonstrate the neural reactivation after MTDS treatment, we examined the number of Ki-67⁺ cells (Ki-67 is a protein produced during cell division) in brain sections after ischemic stroke treatment by immunohistochemistry.

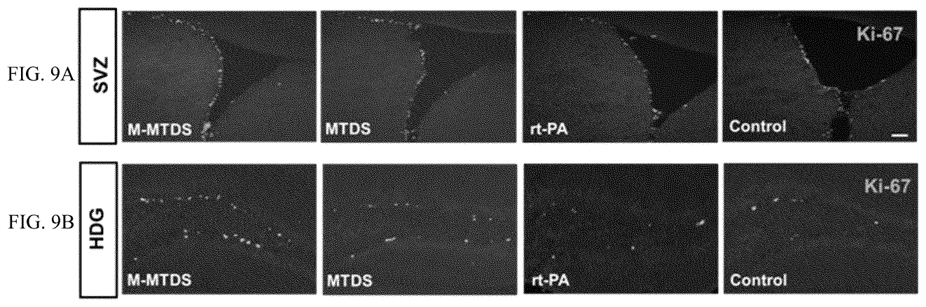

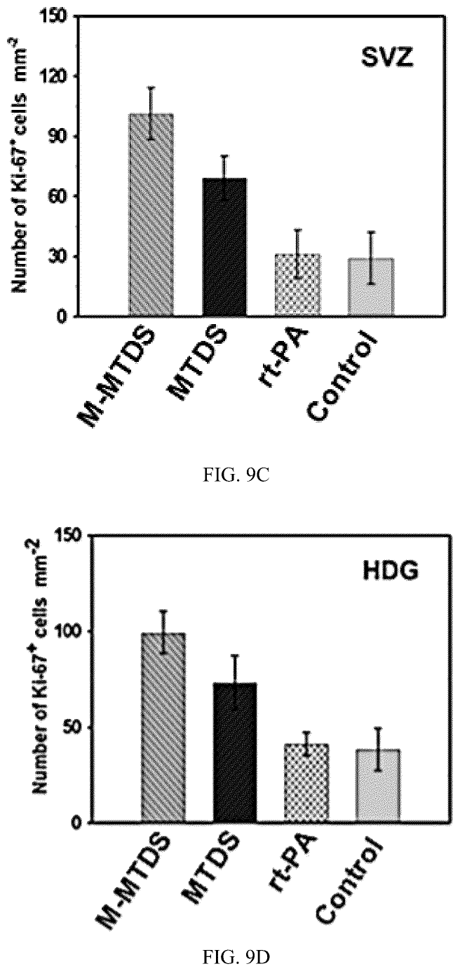

FIGS. 9a and 9b show that there is no significant difference between the rtPA-only administration group and the control group (saline injection). On the contrary, MTDS increased the number of Ki-67⁺ cells more, indicating that the FANC nanocapsule improves the proliferation ability of NPCs. The M-MTDS group, on the other hand, shows the highest number of Ki-67⁺ cells because the MN (magnetic guidance) was able to attract more nanocapsules to accumulate in the stroke affected area. Quantitative data is presented in FIGS. 9c and 9d. In conclusion, the data suggests that MTDS increases the proliferation and activation of NPCs in the ischemic brain. In addition, MN further enhances the effect of MTDS by increasing accumulation and demonstrates the efficacy of the nanocapsules themselves without carrying drugs.

The foregoing description of the exemplary embodiments of the disclosure has been presented only for the purposes of illustration and description and is not intended to be exhaustive or to limit the disclosure to the precise forms disclosed. Many modifications and variations are possible in light of the teaching.

1. A nanocapsule comprising:
a hydrophilic core; and
a hydrophobic shell enclosing the hydrophilic core, wherein the hydrophobic shell contains an outer layer comprising fucoidan, a middle layer comprising carotenoids and metal oxide nanoparticles, and an inner layer comprising fucoidan and contacting the hydrophilic core.

2. The nanocapsule of claim 1, wherein the hydrophilic core comprises tissue plasminogen activator (tPA).

3. The nanocapsule of claim 1, wherein the carotenoids are selected from the group consisting of lycopene, beta and alpha-carotene, lutein, astaxanthin, zeaxanthin, capsanthin, canthaxanthin, phytoene and phytofluene.

4. The nanocapsule of claim 1, wherein the metal oxide nanoparticles are iron oxide nanoparticles.

5. The nanocapsule of claim 4, wherein iron oxide is Fe₃O₄ or Fe₂O₃.

6. The nanocapsule of claim 1, wherein the weight ratio of fucoidan to carotenoids is from 0.55 to 110.

7. The nanocapsule of claim 1, wherein the weight ratio of carotenoids to metal oxide nanoparticles is from 0.01 to 100.

8. The nanocapsule of claim 1, which is free of surface modifier, surfactant, agglomeration inhibitor, dispersion stabilizer and/or viscosity modifier.

9. The nanocapsule of claim 1, wherein the carotenoids and metal oxide nanoparticles form non-covalent bonds.

10. The nanocapsule of claim 1, wherein the carotenoids and fucoidan form hydrogen bonds.

11. The nanocapsule of claim 1, which further comprises iron oxide nanoparticles, gold nanoparticles, gadolinium-based materials or isotopes.

12. A method for treating and/or diagnosing diseases in a subject in need thereof, comprising administering the subject with a therapeutically effective amount of the nanocapsule of claim 1 to the subject.

13. The method of claim 12, wherein the diseases are cancer, thrombosis or hypercoagulation, inflammation, or rheumatoid arthritis.

14. The method of claim 12, wherein the thrombosis or hypercoagulation is atherosclerosis, ischemic stroke, or intracranial hemorrhage.

15. The method of claim 12, wherein the nanocapsule is guided by a magnetic device.

16. The method of claim 12, wherein the hydrophilic core comprises tissue plasminogen activator (tPA).

17. The method of claim 12, wherein the carotenoids are selected from the group consisting of lycopene, beta and alpha-carotene, lutein, astaxanthin, zeaxanthin, capsanthin, canthaxanthin, phytoene and phytofluene.

18. The method of claim 12, wherein the metal oxide nanoparticles are iron oxide nanoparticles.

19. The method of claim 12, wherein the weight ratio of fucoidan to carotenoids is from 0.55 to 110.

20. The method of claim 12, wherein the weight ratio of carotenoids to metal oxide nanoparticles is from 0.01 to 100.
